(19) United States
(12) Patent Application Publication
(10) Pub. No.: US 2006/0159632 A1
(43) Pub. Date: Jul. 20, 2006

(54) Drug formulation for mouth or pharynx comprising local anesthetic

(75) Inventors: Masaki Ishibashi, Higashikagawa-shi (JP); Hidetoshi Hamamoto, Higashikagawa-shi (JP); Keiko Yamasaki, Higashikagawa-shi (JP)

Correspondence Address: BACON & THOMAS, PLLC, 625 SLATERS LANE, FOURTH FLOOR, ALEXANDRIA, VA, 22314, US

(73) Assignee: MEDRX CO., LTD., Higashikagawa-shi (JP)

(21) Appl. No.: 11/331,163

(22) Filed: Jan. 13, 2006

(30) Foreign Application Priority Data
Jan. 20, 2005 (JP) 2005-15479

(51) Int. Cl. A61K 8/46; A61K 31/24; A61K 31/185

(52) U.S. Cl. 424/49; 514/553; 514/536

(57) ABSTRACT

Provided is a drug formulation for mouth or pharynx to be used for medical care or treatment for serious stomatitis caused by a side effect of an anti-tumor agent or the like, and for anesthesia in the pharynx, with remarkably suppressed discomfort due to bitter taste although it contains a local anesthetic as a main efficacious component. The drug formulation of the present invention comprises a local anesthetic as an efficacious component, and a weak acid, its pharmaceutically acceptable salt or a mixture thereof, wherein the mixing ratio of the weak acid to the local anesthetic is 1:1 or more by mole ratio.

### BACKGROUND OF THE INVENTION

1. Field of the Invention

The present invention relates to a drug formulation for mouth or pharynx comprising a local anesthetic.

2. Description of the Related Art

As one of main means for medical care for carcinomas, chemical therapy, that is the administration of synthesized anti-tumor agents, can be exemplified. However, since cancer cells are derived from the normal cells of a patient him- or her-self by malignancy alteration, synthetic anti-tumor agents cause damages also on normal cells. The side effects caused accordingly may be nausea, vomit, alopecia, leukopenia and the like, and it is also known that very serious hardly curable stomatitis is caused.

The stomatitis owing to synthetic anti-tumor agents is caused from the beginning of chemical therapy and is accompanied with dry mouth, abnormal sense of taste and pains. The stomatitis not only causes psychological and physical pains to a patient but also gives secondary burdens on persons in charge of medical care of the patient, for example, doctors and care takers. Further, since the patient loses appetite, it results in worsening of the nutrition and decrease of vigorous power of the patient, and therefore the chemical therapy is sometimes compelled to stop.

To directly moderate the pain from the stomatitis, it is supposed possible to use a local anesthetic. Use of local anesthetics for dental in the interior of mouth has been known. Among local anesthetics, lidocaine is used for anesthesia for pharynx at the time of using a gastocamera. Further, in recent years, it has been known that a drug formulation of lidocaine obtained by gelatification with carrageenan is used to moderate the pain by the stomatitis of a cancer patient.

However, local anesthetics such as lidocaine and ethyl faminobenzoate have unbearable bitter taste, and they are accompanied with strong discomfort for direct application to the mouth of a patent. On the other hand, it is very important to lessen the pain by stomatitis in order to heighten the QOL, quality of life, of a cancer patient.

Besides the therapy for cancer, although a gastocamera is a common medical inspection means, it causes a rather strong pain. Therefore, the previous treatment of pharynx with a local anesthetic is indispensable. However, in spite of its discomfort, the pretreatment requires a patient to keep a local anesthetic in the mouth for 5 minutes or to swallow a bit by a bit for cause the effect on the pharynx. Accordingly, in a medical care field, it has been desired to develop a technique of decreasing the discomfort of local anesthetics.

Local anesthetics are classical drugs and their various formulations have already been known so far. For example, JP-A No. 2001-10977 discloses a composition for oral administration containing an analgesic-antipyretic drug and a local anesthetic. However, the local anesthetic in the composition is contained only for improving the discomfort of the analgesic-antipyretic drug, and the composition contains the analgesic-antipyretic drug, so-called non-steroid type anti-inflammatory anodyne, in an amount of 1 to 10000 times by mass as much as that of the local anesthetic. For the purpose of directly lessening the pain of stomatitis, it is not preferable to use a drug formulation containing a large amount of a non-steroid type anti-inflammatory anodyne causing various side effects such as having gastric mucosal damage. Further, in the case of liquid drug formulations formulated in Examples of JP-A No. 2001-10977, a sodium citrate is added together with a local anesthetic, and therefore it is supposed to be possible to use the formulations as a gargle for mouth and pharynx. However, the local anesthetic contained in the liquid formulations for oral administration is approximately 0.05 to 0.3% only, and with such a small content, it cannot be thought that the local anesthetic as an efficacious component can sufficiently relieve pain from the stomatitis.

JP-A No. 2000-505093 discloses liquid pharmaceutical compositions containing bitter drugs which are improved in the taste. In the publication, lidocaine is exemplified as an example of the bitter drug, and there is a description that citric acid and sodium citrate are normally used. However, lidocaine is simply exemplified together with other drugs, and neither practical drug formulations containing lidocaine are disclosed nor combination use of lidocaine and citric acid or so is described. Further, with respect to the drug formulations in the cited document, the component to suppress the bitter taste is polyvinylpyrrolidone or the like, and citric acid is used only for adjusting and keeping the pH value. Accordingly, the amount of citric acid or the like added to Examples of JP-A No. 2000-505093 is slight and it cannot be thought to be possible to suppress the discomfort of the bitter drug by only citric acid or the like. Further, the drug formulations of the cited document are intended to swallow, and use of the drug formulations in mouth or pharynx is not at all described or suggested.

U.S. Pat. No. 4,041,174 discloses therapeutic methods for depression by administration of a local anesthetic, and there is a description that an organic weak acid component may be added to the therapeutic drug formulations. However, the drug formulations of the cited document are for therapy of depression and therefore, there is of course no description of use them for the stomatitis in mouth or pharynx. Further, there is no description relevant to the amount of the organic weak acid component to be added. Only a drug formulation, Gerovital H3, obtained by dissolving 100 mg of procaine hydrochloride together with 6 mg of benzoic acid, 5 mg of potassium pyrosulfite (K₂S₂O₅) and 0.5 mg of sodium secondary phosphate (Na₂HPO₄) in 5 cc of water is described. However, the total mole number these three acids is about 0.075 mmol while the mole number of procaine hydrochloride is about 0.37 mmol. With such a small amount of the acids, it cannot be thought that the effect of decreasing the bitter taste of the local anesthetic is sufficient.

### BRIEF SUMMARY OF THE INVENTION

As described above, drug formulations containing a local anesthetic have already been known. However, there is neither formulation aiming for directly relieving pains from the stomatitis in mouth among them nor formulation with sufficiently suppressed discomfort of anesthetics in the case of direct use in mouth or pharynx.

Accordingly, the problems to be solved by the invention is to provide a drug formulation for mouth or pharynx to be used for medical care or treatment for serious stomatitis caused by a side effect of an anti-tumor agent and the like, or for anesthesia in pharynx, with remarkably suppressed discomfort due to the bitter taste although it contains a local anesthetic as a main efficacious component.

To solve the above problemsms, the present inventors have repeatedly made investigations of drug formulation components capable of suppressing bitter taste even in the case a local anesthetic is applied directly to mouth or pharynx but not temporarily passed as an oral drug formulation. Consequently, the inventors have found that the bitter taste of local anesthetics can remarkably be lessened by adding a proper amount of a weak acid, and accordingly have accomplished the invention.

The drug formulation for mouth or pharynx of the present invention comprising:

a local anesthetic as an efficacious component, and

a weak acid, its pharmaceutically acceptable salt, or a mixture thereof,

wherein the mixing ratio of the weak acid to the local anesthetic is 1:1 or more by mole ratio.

The above-mentioned drug formulation is preferable to contain 1.0% by mass or more of the local anesthetic. Since the main efficacious component of the drug formulation of the present invention is the local anesthetic, if it is less than 1.0% by mass, the effect of the medical care or treatment may possibly be insufficient. Also, the formulation further containing sodium azulenesulfonate is preferable. Sodium azulenesulfonate is not only already used for medical care for stomatitis but also has a function of lessening the bitter taste of local anesthetics based on the finings of the inventors.

The weak acid to be added to the drug formulation of the invention is preferably an organic acid, and more preferably one or more acids selected from the group consisting of malic acid, citric acid, tartaric acid and the salt thereof. As the local anesthetic is preferably lidocaine or its pharmaceutically acceptable salts.

The drug formulation of the invention is preferable to be liquid and to be used as a gargle. It is because it can be used conveniently and excellent in the immediate efficacy.

Pain relief of serious stomatitis caused by a side effect of a synthetic anti-tumor agent is a very important issue for a cancer patient receiving chemical therapy against cancer in order to improve QOL. It has been known that a local anesthetic is effective for such pain relief. Also, the local anesthetic is sometimes used directly for anesthesia for pharynx. However, a local anesthetic cause strong harm to a patient if it is directly applied to the affected part of the stomatitis owing to the unbearable bitter taste. Therefore, in a medical therapeutic work field, it has been desired to develop a technique of suppressing the bitter taste of local anesthetics.

Application of the invention makes it possible to remove the pain from the patient at the time of direct administration of a local anesthetic to the mouth and the pharynx. Accordingly, the drug formulation of the invention for mouth or pharynx is very valuable in industrial fields for not only relieving the pain of a cancer patient but also satisfying the requirement in the medical therapeutic work field.

### DETAILED DESCRIPTION OF THE INVENTION

The drug formulation of the present invention can be applied directly to mouth and pharynx, and comprises a local anesthetic as an efficacious component, and a weak acid, its pharmaceutically acceptable salt or a mixture thereof, wherein the mixing ratio of the weak acid to the local anesthetic is 1:1 or more by mole ratio.

The kind of local anesthetic of the invention is not particularly limited if it can works on the peripheral neurons and directly lessen the pain from the stomatitis, or anesthetize a local part. The examples of the local anesthetic are lidocaine, ethyl p-aminobenzoate, oxybuprocaine, tetracaine, procaine, diethylaminoethyl p-butylaminobenzoate and their pharmaceutically acceptable salts, which may include their mixtures. Particularly preferable examples are its lidocaine and pharmaceutically acceptable salts.

The local anesthetic of the invention is a main efficacious component for directly lessening the pain from the stomatitis and anesthetizing a local part. Accordingly, it is preferable to be contained in an amount of 1.0% by mass or more, more preferably 1.5% by mass or more, and even more preferably 1.7% by mass or more in the drug formulation. Also, since the invention aims to directly lessen the pain from the stomatitis by the local anesthetic, the local anesthetic is a main efficacious component and any drug other than the local anesthetic is at least prevented from addition more than the local anesthetic. However, any drug other than the local anesthetic may be added for assistance. For example, a non-steroid type anti-inflammatory anodyne is inhibited from addition more than the local anesthetic.

The weak acid to be used in the invention may include preferably those having an acid dissociation constant, PKa=−log Ka, of 2 or more and regardless of inorganic acids or organic acids, any weak acids can be usable. Examples of the inorganic weak acids are phosphoric acid, carbonic acid and salts thereof. Examples of the organic acids are malic acid, citric acid, tartaric acid, ascorbic acid, succinic acid, fumaric acid, maleic acid, gluconic acid, glucuronic acid and salts thereof, and preferable organic acids may be malic acid, citric acid, tartaric acid and salts thereof. Also, examples of preferable weak acid may include divalent or trivalent weak acids of malic acid, citric acid, tartaric acid and phosphoric acid. Regardless of inorganic acids and organic acids, their mixtures may be used. Citric acid is particularly preferably used.

The mixing ratio of the weak acid to the local anesthetic, weak acid/local anesthetic, is preferably 1:1 or more by mole. That is because it can be said that the mixing ratio of the weak acid is more, the bitter taste of the local anesthetic can be suppressed more, and if the mole ratio is less than 1, the discomfort cannot sufficiently be lessened in some cases. The mole ratio is preferably 1:1.2 or more, 1:1.4 or more, 1:1.6 or more, and 1:2 or more. In this connection, if the mixing ratio of the weak acid is too high, the mixing ratio of the local anesthetic is relatively decreased, and the efficacy of the drug may possibly become insufficient. Therefore, it is preferable to adjust the ratio to be 1:5 or less.

Sodium azulenesulfonate may be added to the drug formulation of the invention. Sodium azulenesulfonate is a non-steroid type anti-inflammatory showing direct local anti-inflammatory effect on the inflamed tissues, and not only used for medical care for gastritis and gastric ulcer but also used for stomatitis as an efficacious component for a gargle. In the invention, sodium azulenesulfonate is added for a purpose of auxiliary suppression of the bitter taste of the local anesthetic. Accordingly, the addition amount is no need to be so much to exhibit the efficacy and may be about 0.1 to 1% by mass to the local anesthetic. In this connection, sodium azulenesulfonate belongs to a different category from that of a non-steroid type analgesic anti-inflammatory drug showing the antipyretic effect. Also, sodium azulenesulfonate promotes curing of the damaged gastric mucosa or protects the stomach by suppressing activity of pepsin. From this aspect, it can be distinguished from the non-steroid type analgesic anti-inflammatory drug which tends to damage the gastric mucosa.

The drug formulation of the invention may contain other additive components without departing from the spirit and scope of the invention. For example, disinfectants such as polyvinyl pyrrolidone, benzalkonium chloride, chlorhexidine hydrochloride; steroids such as Triamcinolone Acetonide, dexamethasone; antibiotics; antifungal agent; and the like. Besides, sweeteners may be added. Sweeteners to be used in the invention may include aspartame, saccharine, sodium saccharine, stevia, thaumatin, erythritol, sorbitol, xylitol, glycerin and dipotassium glycyrrhizinate and also their mixtures. Preferable sweeteners are aspartame, saccharine, sodium saccharine and stevia.

Besides, flavors may be added. The flavors to be used in the invention may be those of lemon, orange, grapefruit, pineapple, banana, chocolate, yogurt, vanilla, menthol and the like. By the addition of such flavors, more favorable feeling under the administration can be obtained.

Further, common additives for drug formulations, which are pharmaceutically acceptable, harmless and inactive additives, may be added. These additives may be, for example, fillers such as corn starch, potato starch, refined sucrose, mannitol, xylitol, sorbitol, talc, kaolin, calcium hydrogen phosphate, calcium sulfate, calcium carbonate and crystalline cellulose; lubricants such as magnesium stearate and calcium stearate; disintegrators such as calcium carboxymethyl cellulose, sodium carboxymethyl cellulose and hydroxymethyl cellulose with low substitution degree; binders such as polyethylene glycol, hydroxypropyl cellulose, hydroxypropyl methyl cellulose, polyvinylpyrrolidone, gelatin, methyl cellulose, gum arabic powder and polyvinyl alcohol; in addition, thickeners, coloring agents, taste amendments, adsorbents, preservatives, stabilizers, moisturizing agents, antistatic agents and pH adjustment agents.

The form of the drug formulation of the invention may be a liquid agent, an ointment, a jelly agent, a gumi agent and dry syrup, and may be produced by conventional methods. The liquid agent is produced by dissolving or suspending the above-mentioned components in purified water or distilled water. The liquid agent may be kept in a mouth until the pharmaceutical efficacy such as lessening the pain from the stomatitis is exhibited, and may be used as a gargle. Such a gargle has an advantageous point that it is easily made available and excellent in immediate efficacy. The dry syrup can be used as the liquid agent by being dissolved in water. The ointment may be used while being applied directly to the stomatitis.

The jelly agent can cause a local surface anesthetizing effect by being applied directly to the affected part of the stomatitis. As a result, different from a liquid agent or the like which causes an effect on the entire part with which the drug is brought into contact, the possibility of anesthetizing effect on the part other than a needed part can be lowered. Thus an incident of biting the interior of mouth at the time of taking meal after administration of the drug formulation can be suppressed as much as possible.

### EXAMPLES

Although the invention will be described more in detail with reference to examples, it is not intended that the invention be limited to the illustrated examples. The values of the addition amounts in the examples are all on the basis of % by mass.

### Test Example 1

According to the mixing ratios shown in Table 1, liquid drug formulations, sample liquids 1 to 15, containing lidocaine hydrochloride as a local anesthetic were produced. The mole ratio in the following is mole ratio of the weak acid to the local anesthetic, that is weak acid/local anesthetic.

TABLE 1

| | Sample No. | | | | | | | | | | | | | | |
|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 |
| Lidocaine | 2.0 | 2.0 | 2.0 | 2.0 | 2.0 | 2.0 | 2.0 | 2.0 | 2.0 | 2.0 | 2.0 | 2.0 | 2.0 | 2.0 | 2.0 |
| hydrochloride | | | | | | | | | | | | | | | |
| Citric acid | 0.45 | 0.73 | 1.45 | 2.9 | 4.5 | 0.5 | 1.3 | | | | | | | | |
| Sodium citrate | | | | | | 0.3 | 0.4 | | | | | | | | |
| Phosphoric acid | | | | | | | | 0.21 | 0.34 | 0.68 | 1.35 | 2.1 | 0.3 | 0.63 | |
| Sodium dihydrogen | | | | | | | | | | | | | 0.1 | 0.6 | |
| phosphate | | | | | | | | | | | | | | | |
| Purified water | Bal- | Bal- | Bal- | Bal- | Bal- | Bal- | Bal- | Bal- | Bal- | Bal- | Bal- | Bal- | Bal- | Bal- | Bal- |
| | ance | ance | ance | ance | ance | ance | ance | ance | ance | ance | ance | ance | ance | ance | ance |
| Mole ratio | 0.3 | 0.5 | 1.0 | 2.0 | 3.0 | 0.5 | 1.0 | 0.3 | 0.5 | 1.0 | 2.0 | 3.0 | 0.5 | 1.5 | — |

Using the sample liquids 1 to 15 produced according to Table 1 were subjected to a sensory test by 10 healthy examinees. About 1 mL of each sample liquid was kept in the mouth and then discharged, examinees were left still for 30 seconds without gargling and the distasteful aftertaste thereafter was determined. The number of the examinees “feeling no bitter taste”, “feeling slight bitter taste” or “feeling bitter taste” is in Table 2.

TABLE 2

| | | Feeling | Feeling slight | Feeling |
|---|---|---|---|---|
| No. | Mole ratio | no bitter taste | bitter taste | bitter taste |
| 1 | 0.3 | 0 | 0 | 10 |
| 2 | 0.5 | 0 | 1 | 9 |
| 3 | 1.0 | 8 | 2 | 0 |
| 4 | 2.0 | 10 | 0 | 0 |
| 5 | 3.0 | 10 | 0 | 0 |
| 6 | 0.5 | 0 | 2 | 8 |
| 7 | 1.0 | 9 | 1 | 0 |
| 8 | 0.3 | 0 | 0 | 10 |
| 9 | 0.5 | 0 | 1 | 9 |
| 10 | 1.0 | 6 | 3 | 1 |
| 11 | 2.0 | 8 | 2 | 0 |
| 12 | 3.0 | 10 | 0 | 0 |
| 13 | 0.5 | 0 | 2 | 8 |
| 14 | 1.5 | 8 | 2 | 0 |
| 15 | — | 0 | 0 | 10 |

From the above shown results, the local anesthetic-containing drug formulations containing citric acid or phosphoric acid or their salt as a weak acid are proved having suppressed distasteful bitter taste as compared with the case containing no weak acid, the sample liquid 15. Also, such a bitter taste lessening effect depends on the mole ratio of the weak acid and the local anesthetic, and it is particularly effective if the mole ratio of the weak acid to the local anesthetic is 1:1 or more.

### Test Example 2

According to the mixing ratios shown in Table 3, liquid drug formulations containing lidocaine as a local anesthetic, sample liquids 16 to 25, were produced.

TABLE 3

| | Sample No. | | | | | | | | | |
|---|---|---|---|---|---|---|---|---|---|---|
| | 16 | 17 | 18 | 19 | 20 | 21 | 22 | 23 | 24 | 25 |
| Lidocaine | 1.73 | 1.73 | 1.73 | 1.73 | 1.73 | 1.73 | 1.73 | 1.74 | 1.75 | 1.76 |
| Citric acid | 0.49 | 0.5 | 0.51 | 0.52 | 0.52 | 0.52 | 0.52 | | | |
| Sodium citrate | 0.05 | 0.38 | 1.03 | 1.45 | 2.0 | 3.6 | 5.8 | | | |
| Phosphoric acid | | | | | | | | 0.2 | 0.35 | 0.82 |
| Sodium dihydrogen | | | | | | | | 0.3 | 0.6 | 1.0 |
| phosphate | | | | | | | | | | |
| Glycerin | 13.3 | 13.3 | 13.3 | 13.3 | 13.3 | 13.3 | 13.3 | 13.4 | 13.5 | 13.6 |
| Concentrated | 4.2 | 4.2 | 4.2 | 4.2 | 4.2 | 4.2 | 4.2 | 4.3 | 4.4 | 4.5 |
| glycerin | | | | | | | | | | |
| Macrogol 400 | 5.1 | 5.1 | 5.1 | 5.1 | 5.1 | 5.1 | 5.1 | 5.2 | 5.3 | 5.4 |
| Propylene glycol | 4.6 | 4.6 | 4.6 | 4.6 | 4.6 | 4.6 | 4.6 | 4.7 | 4.8 | 4.9 |
| Preservative | Proper | Proper | Proper | Proper | Proper | Proper | Proper | Proper | Proper | Proper |
| | amount | amount | amount | amount | amount | amount | amount | amount | amount | amount |
| Purified water | Balance | Balance | Balance | Balance | Balance | Balance | Balance | Balance | Balance | Balance |
| Mole ratio | 0.33 | 0.5 | 0.8 | 1 | 1.25 | 2 | 3 | 0.5 | 1 | 2 |

Using the sample liquids 16 to 25 produced according to Table 3, which are liquid formulations containing the local anesthetic, were subjected to a sensory test by 10 healthy examinees in the same manner as the above-mentioned Test Example 1. The results are shown in Table 4.

TABLE 4

| | Feeling | Feeling slight | Feeling |
|---|---|---|---|
| No. | no bitter taste | bitter taste | bitter taste |
| 16 | 0 | 0 | 10 |
| 17 | 0 | 1 | 9 |
| 18 | 0 | 1 | 9 |
| 19 | 3 | 6 | 1 |
| 20 | 7 | 3 | 0 |
| 21 | 9 | 1 | 0 |
| 22 | 10 | 0 | 0 |
| 23 | 0 | 0 | 10 |
| 24 | 6 | 3 | 1 |
| 25 | 9 | 1 | 0 |

From the above shown results, the local anesthetic-containing drug formulations containing a weak acid are proved having suppressed bitter taste of the local anesthetic. Also, the mole ratio of the weak acids to the local anesthetic at which the distasteful bitter taste is lessened is same as the result of Test Example 1. That is, a particularly significant bitter taste lessening effect is caused in the case of the formulations having a mole ratio of the weak acids to the local anesthetic of 1:1 or more.

### Test Example 3

According to the mixing ratios shown in Table 5, liquid drug formulations, a sample liquid 26 containing lidocaine and sodium azulenesulfonate, and a sample liquid 27 containing lidocaine but no sodium azulenesulfonate were produced.

TABLE 5

No.

| | 26 | 27 |
|---|---|---|
| Lidocaine | 1.73 | 1.73 |
| Citric acid | 0.52 | 0.52 |
| Sodium citrate | 2.0 | 2.0 |
| Glycerin | 13.3 | 13.3 |
| Concentrated glycerin | 4.2 | 4.2 |
| Macrogol 400 | 5.1 | 5.1 |
| Propylene glycol | 4.6 | 4.6 |
| Sodium azulenesulfonate | 0.01 | — |
| Preservative | Proper amount | Proper amount |
| Purified water | Balance | Balance |
| Mole ratio | 1.25 | 1.25 |

Using the sample liquids 26 to 27 containing the local anesthetic and produced according to the mixing ratios in Table 5 were subjected to a sensory test in the same manner as the above-mentioned Test Example 1. The results are shown in Table 6.

TABLE 6

| | Feeling | Feeling slight | Feeling |
|---|---|---|---|
| No. | no bitter taste | bitter taste | bitter taste |
| 26 | 9 | 1 | 0 |
| 27 | 1 | 6 | 3 |

From the above shown results, it is proved that addition of sodium azulenesulfonate to the drug formulation of the invention further lessen the distasteful aftertaste.

### Test Example 4

According to the mixing ratios shown in Table 7, jelly formulations containing lidocaine as local anesthetic, drug formulations 28 to 30, were produced.

TABLE 7

| | No. | | |
|---|---|---|---|
| | 28 | 29 | 30 |
| Lidocaine hydrochloride | 2 | 2 | 2 |
| Citric acid | 0.05 | 0.07 | 0.09 |
| Sodium citrate | 1 | 2 | 4.5 |
| Sodium saccharine | 0.5 | 0.5 | 0.5 |
| Sodium azulenesulfonate | 0.008 | 0.008 | 0.008 |
| Dipotassium glycyrrhizinate | 0.05 | 0.05 | 0.05 |
| Polyvinyl alcohol | 0.3 | 0.3 | 0.3 |
| Polyethylene glycol | 5.1 | 5.1 | 5.1 |
| Glycerin | 13.3 | 13.3 | 13.3 |
| Concentrated glycerin | 4.2 | 4.2 | 4.2 |
| Propylene glycol | 4.6 | 4.6 | 4.6 |
| Carrageenan | 0.7 | 0.7 | 0.7 |
| Xanthane gum | 0.3 | 0.3 | 0.3 |
| Agar powder | 0.17 | 0.17 | 0.17 |
| Carob bean gum | 0.05 | 0.05 | 0.05 |
| Preservative | Proper | Proper | Proper |
| | amount | amount | amount |
| Purified water | Balance | Balance | Balance |
| Mole ratio | 0.5 | 1 | 2.2 |

Using the jelly formulations 28 to 30 produced by mixing the components at the ratios according to Table 7 were subjected to a sensory test by 10 healthy examinees based on the sense of taste in the case that 5 g of each formulation was kept and bitted in the mouth. The results are shown in Table 8.

TABLE 8

| | Feeling | Feeling slight | Feeling |
|---|---|---|---|
| No. | no bitter taste | bitter taste | bitter taste |
| 28 | 0 | 4 | 6 |
| 29 | 8 | 2 | 0 |
| 30 | 10 | 0 | 0 |

From the above shown results, it is proved that the effect of addition of weak acids on lessening the bitter taste of the local anesthetic is effective not only in the case of the liquid formulations but also in the case of the jelly formulations.

### Test Example 5

According to the mixing ratios shown in Table 9, ointment formulations containing lidocaine were produced.

TABLE 9

| | No. | | |
|---|---|---|---|
| | 31 | 32 | 33 |
| Lidocaine | 2 | 2 | 2 |
| Citric acid | 1 | 2 | 4 |
| Sodium saccharine | 0.5 | 0.5 | 0.5 |
| Isopropyl myristate | 10 | 10 | 10 |
| Sodium carmellose | 10 | 10 | 10 |
| Polyvinyl alcohol | 2 | 6 | 6 |
| Carboxyvinyl polymer | 1 | 1 | 1 |
| Polyethylene glycol | 20 | 20 | 20 |
| Concentrated glycerin | 40 | 40 | 40 |
| Preservative | Proper amount | Proper amount | Proper amount |
| Purified water | Balance | Balance | Balance |
| Mole ratio | 0.5 | 1 | 2.2 |

Using the ointment formulations 31 to 33 produced by mixing the components at the ratios according to Table 9 were subjected to a sensory test by 10 healthy examinees. About 0.5 g of each formulation was applied to the interior of the mouth and the distasteful feeling owing to the bitter taste after 30 seconds was determined. The results are shown in Table 10.

TABLE 10

| | Feeling | Feeling slight | Feeling |
|---|---|---|---|
| No. | no bitter taste | bitter taste | bitter taste |
| 31 | 0 | 8 | 2 |
| 32 | 9 | 1 | 0 |
| 33 | 10 | 0 | 0 |

From the above shown results, it is proved that the effect of addition of weak acids on lessening the bitter taste of the local anesthetic is effective also in the case of the ointment formulations. Further, the same result as those of the case of liquid formulations and jelly formulations was obtained regarding the mole ratio of the weak acids to the local anesthetic, by which the bitter taste distacomfort by the local anesthetic is lessened. That is, in the formulations having the mole ratio of the weak acid to the local anesthetic at 1:1 or more, an effect particularly effective for lessening bitter taste is caused.

### Formulation Example 1

Lidocaine-Containing Ointment Formulation

A 5% lidocaine ointment formulation was produced by kneading lidocaine 2.0, citric acid 2.5, sodium saccharine 2.0, L-menthol 0.02, isopropyl myristate 10.0, sodium carboxymethyl cellulose 10.0, polyvinyl alcohol 2.0, carboxyvinyl polymer 1.0, polyethylene glycol 20.0, concentrated glycerin 40.0 and a preservative in a proper amount with purified water adjusted to make the total amount 100.

### Formulation Example 2

Lidocaine-Containing Ointment Formulation

A 5% lidocaine ointment formulation was produced by kneading lidocaine 2.0, DL-malic acid 2.0, sodium saccharine 2.0, L-menthol 0.02, polyvinyl alcohol 2.0, carboxyvinyl polymer 1.0, polyethylene glycol 20.0, concentrated glycerin 40.0 and a preservative in a proper amount with purified water adjusted to make the total amount 100.

### Formulation Example 3

Ethyl p-Aminobenzoate-Containing Ointment Formulation

A 30% ethyl p-aminobenzoate ointment formulation was produced by kneading ethyl p-aminobenzoate 3.0, citric acid 5.0, sodium saccharine 2.0, L-menthol 0.02, sodium carboxymethyl cellulose 18.0, white sucrose 7.5, polyethylene glycol 20.0, carrageenan 7.5 and a preservative in a proper amount with purified water adjusted to make the total amount 100.

### Formulation Example 4

Lidocaine-Containing Liquid Formulation, Gargle

A 2% lidocaine-containing liquid formulation was produced by dissolving or suspending lidocaine 2.0, citric acid 0.5, sodium citrate 1.66, sodium saccharine 0.1, sodium azulenesulfonate 0.01, polyvinyl alcohol 0.3, polyethylene glycol 5.1, glycerin 13.3, concentrated glycerin 4.2, propylene glycol 4.6 and a proper amount of a preservative in purified water adjusted to make the total amount 100.

### Formulation Example 5

Lidocaine Hydrochloride-Containing Jelly Formulation

A 2% lidocaine hydrochloride-containing jelly formulation was produced by dissolving or suspending lidocaine hydrochloride 2.0, citric acid 0.09, sodium citrate 4.5, sodium saccharine 0.5, sodium azulenesulfonate 0.008, dipotassium glycyrrhizinate 0.5, polyvinyl alcohol 0.3, polyethylene glycol 5.1, glycerin 13.3, concentrated glycerin 4.2, propylene glycol 4.6, carrageenan 0.7, xanthane gum 0.3, agar powder 0.17, carob bean gum 0.05 and a proper amount of a preservative in purified water adjusted to make the total amount 100, heating to dissolve the mixture and then cooling the mixture to a room temperature.

What is claimed is:

1. A drug formulation for mouth or pharynx comprising:
a local anesthetic as an efficacious component, and
a weak acid, its pharmaceutically acceptable salt or a mixture thereof,
wherein the mixing ratio of the weak acid to the local anesthetic is 1:1 or more by mole ratio.

2. The drug formulation according to claim 1, comprising 1.0 by mass or more of the local anesthetic.

3. The drug formulation according to claim 1, further comprising a sodium azulenesulfonate.

4. The drug formulation according to claim 2, further comprising a sodium azulenesulfonate.

5. The drug formulation according to claim 1, wherein the weak acid is an organic acid.

6. The drug formulation according to claim 1, wherein the weak acid is one or more acids selected from the group consisting of a malic acid, a citric acid, a tartaric acid and the salt thereof.

7. The drug formulation according to claim 1, wherein the local anesthetic is a lidocaine or its pharmaceutically acceptable salts.

8. The drug formulation according to claim 1, being liquid and to be used as a gargle.
